(19) United States
(12) Patent Application Publication
(10) Pub. No.: US 2022/0168213 A1
(43) Pub. Date: Jun. 2, 2022

(54) WATER-IN-OIL EMULSION COMPOSITION

(71) Applicant: GREENVINES BIOTECH CO., LTD., Taipei City (TW)

(72) Inventors: Yi-Wen LIAO, Taipei City (TW); Jie LI, Taipei City (TW)

(21) Appl. No.: 17/108,361

(22) Filed: Dec. 1, 2020

(51) Int. Cl. A61K 8/9794; A61K 8/06; A61K 8/34; A61K 8/42; A61K 8/99; A61K 8/9789; A61K 8/365; A61K 8/362; A61K 8/368; A61Q 17/04; A61K 8/25; A61K 8/29; A61K 8/36; A61K 8/20

(52) U.S. Cl. CPC A61K 8/9794; A61K 8/20; A61K 8/342; A61K 8/42; A61K 8/99; A61K 8/9789; A61K 8/365; A61K 8/362; A61K 8/368; A61K 8/345; A61Q 17/04; A61K 8/25; A61K 8/29; A61K 8/361; A61K 8/064

(57) ABSTRACT

Disclosed herein is a water-in-oil emulsion composition that includes, based on the total weight of the water-in-oil emulsion composition, 2 wt % to 10 wt % of Avena sativa kernel oil, 15 wt % to 38 wt % of octyldodecanol, and water. The water-in-oil emulsion composition is free from a surfactant and a gelling agent.

### FIELD

The disclosure relates to a water-in-oil emulsion composition and more particularly to a surfactant-free and gelling agent-free emulsion composition.

### BACKGROUND

Water-in-oil emulsions have a high water or aqueous phase content and are particularly suitable for certain uses, because they spread easily and evenly, forming a long-lasting, continuous, protective film on the skin, and also mimic the skin's natural lipid barrier. Such characteristics are important in products that are desirably water-proof and/or long-wearing, e.g., barrier lotions, moisturizing lotions, and sunscreens.

Based on the nature of water-in-oil emulsions, in which two mutually insoluble or nearly insoluble phases are dispersed one within the other, a surfactant and a gelling agent must be added to avoid separation of the emulsions into oil and aqueous layers. However, the surfactant and the gelling agent increase the stickiness feeling of the water-in-oil emulsions during use and might cause skin irritation. In addition, use of surfactants and/or gelling agents in the water-in-oil emulsions might incur a higher manufacturing cost.

In view of the foregoing, there is still a need to develop a new water-in-oil emulsion that does not contain a surfactant and a gelling agent but can retain a high amount of an oil component.

### SUMMARY

Accordingly, the present disclosure provides a water-in-oil emulsion composition including, based on the total weight of the water-in-oil emulsion composition, 2 wt % to 10 wt % of Avena sativa kernel oil, 15 wt % to 38 wt % of octyldodecanol, and water. The water-in-oil emulsion composition is free from a surfactant and a gelling agent.

### DETAILED DESCRIPTION

It is to be understood that, if any prior art publication is referred to herein, such reference does not constitute an admission that the publication forms a part of the common general knowledge in the art, in Taiwan or any other country.

For the purpose of this specification, it should be clearly understood that the word “comprising” means “including but not limited to”, and that the word “comprise” has a corresponding meaning.

Unless otherwise defined, all technical and scientific terms used herein have the meaning as commonly understood by a person skilled in the art to which the present disclosure belongs. One skilled in the art will recognize many methods and materials similar or equivalent to those described herein, which could be used in the practice of the present disclosure. Indeed, the present disclosure is in no way limited to the methods and materials described.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value, can be meant to encompass variations of, in some aspects ±100%, in some aspects ±50%, in some aspects ±20%, in some aspects ±10%, in some aspects ±5%, in some aspects ±1%, in some aspects ±0.5%, and in some aspects ±0.1% from the specified amount, as such variations are appropriate to perform the disclosed methods or employ the disclosed compositions.

The present disclosure provides a water-in-oil emulsion composition including, based on the total weight of the water-in-oil emulsion composition, 2 wt % to 10 wt % of Avena sativa kernel oil, 15 wt % to 38 wt % of octyldodecanol, and water. The water-in-oil emulsion composition is free from a surfactant and a gelling agent.

According to the present disclosure, the Avena sativa kernel oil may include 0.5 wt % to 2 wt % of a ceramide, based on the total weight of the Avena sativa kernel oil.

In certain embodiments, the Avena sativa kernel oil may include 0.7 wt % to 1.15 wt % of a ceramide, based on the total weight of the Avena sativa kernel oil.

According to the present disclosure, the water-in-oil emulsion composition may further include a preservative.

As used herein, the term “preservative” refers to any component that is commonly added into an emulsion composition to inhibit the growth of microorganisms.

Examples of the preservative suitable for use in this disclosure may include, but are not limited to, Lactobacillus/radish root ferment filtrate, levulinic acid, sodium levulinate, sodium citrate, sodium anisate, glycerol, and combinations thereof.

According to the present disclosure, the preservative may be present in an amount ranging from 2 wt % to 4 wt %, based on the total weight of the water-in-oil emulsion composition.

In an exemplary embodiment of the present disclosure, the water-in-oil emulsion composition may include 2 wt % to 5 wt % of the Avena sativa kernel oil, 15 wt % to 18 wt % of octyldodecanol, 4 wt % of pentylene glycol, and 76 wt % of water, based on the total weight of the water-in-oil emulsion composition.

In another exemplary embodiment of the present disclosure, the water-in-oil emulsion composition may include 2 wt % to 8 wt % of the Avena sativa kernel oil, 22 wt % to 28 wt % of octyldodecanol, 4 wt % of pentylene glycol, and 66 wt % of water, based on the total weight of the water-in-oil emulsion composition.

In yet another exemplary embodiment of the present disclosure, the water-in-oil emulsion composition may include 2 wt % to 10 wt % of the Avena sativa kernel oil, 30 wt % to 38 wt % of octyldodecanol, 4 wt % of pentylene glycol, and 56 wt % of water, based on the total weight of the water-in-oil emulsion composition.

According to the present disclosure, the water-in-oil emulsion composition may be formulated into a form of a skincare product using technology well known to those skilled in the art. The suitable form includes, but is not limited to, day creams, night creams, care creams, nutrient creams, cleansing milks, makeup remover lotions, skin milks, sunscreen lotions, skin protection creams, body lotions, ointments, hand creams, and the like.

Therefore, the present disclosure also provides a sunscreen lotion, which includes a water-in-oil emulsion composition as described above.

In an exemplary embodiment of the present disclosure, the sunscreen lotion may include 7 wt % of Avena sativa kernel oil, 25 wt % of octyldodecanol, 10 wt % of titanium dioxide coated with alumina and stearic acid, 2 wt % of iron oxide and silicon dioxide-titanium dioxide mixed oxide, 50.47 wt % of water, 3 wt % of pentylene glycol, 2 wt % of a preservative (containing glycerin, water, sodium levulinate, and sodium anisate), 0.03 wt % of citric acid, and 0.5 wt % of sodium chloride, based on the total weight of the sunscreen lotion.

This disclosure will be further described by way of the following examples. However, it should be understood that the following examples are solely intended for the purpose of illustration and should not be construed as limiting the disclosure in practice.

### EXAMPLES

### Example 1. Preparation of Water-In-Oil Emulsion Composition of Present Disclosure

In this experiment, the applicant tested 24 water-in-oil emulsion compositions, including eighteen comparative compositions and six exemplary compositions. Each water-in-oil emulsion composition was prepared using the respective recipe shown in Table 1, and the preparation process of these water-in-oil emulsion compositions was described as follows.

The intended components of the oil phase were homogeneously mixed under stirring, so as to obtain a first mixture. The intended components of the aqueous phase were homogeneously mixed, so as to obtain a second mixture. The second mixture was divided into 20 equal parts, and all the equal parts were then added into the first mixture in batches under stirring, so as to obtain a water-in-oil emulsion composition.

TABLE 1

| | Oil phase | | | | | | |
|---|---|---|---|---|---|---|---|
| | | | Hydrogenated | | | | |
| | | | ethylhexyl | | | | |
| | Avena | Helianthus | olivate and | | | | |
| | sativa | annuus | hydrogenated | | | Aqueous phase | |
| | kernel | (sunflower) | olive oil | Liquid | | | Pentylene |
| | oil | seed oil | unsaponifiables | paraffin | Octyldodecanol | Water | glycol |
| | Amount (wt %) | | | | | | |
| Exemplary composition | 2 | — | — | — | 18 | 76 | 4 |
| 1-a1 | | | | | | | |
| Exemplary composition | 5 | — | — | — | 15 | 76 | 4 |
| 1-a2 | | | | | | | |
| Comparative composition | — | 2 | — | — | 18 | 76 | 4 |
| 1-a1 | | | | | | | |
| Comparative composition | — | 5 | — | — | 15 | 76 | 4 |
| 1-a2 | | | | | | | |
| Comparative composition | — | — | 2 | — | 18 | 76 | 4 |
| 2-a1 | | | | | | | |
| Comparative composition | — | — | 5 | — | 15 | 76 | 4 |
| 2-a2 | | | | | | | |
| Comparative composition | — | — | — | 2 | 18 | 76 | 4 |
| 3-a1 | | | | | | | |
| Comparative composition | — | — | — | 5 | 15 | 76 | 4 |
| 3-a2 | | | | | | | |
| Exemplary composition | 2 | — | — | — | 28 | 66 | 4 |
| 1-b1 | | | | | | | |
| Exemplary composition | 8 | — | — | — | 22 | 66 | 4 |
| 1-b2 | | | | | | | |
| Comparative composition | — | 2 | — | — | 28 | 66 | 4 |
| 1-b1 | | | | | | | |
| Comparative composition | — | 8 | — | — | 22 | 66 | 4 |
| 1-b2 | | | | | | | |
| Comparative composition | — | — | 2 | — | 28 | 66 | 4 |
| 2-b1 | | | | | | | |
| Comparative composition | — | — | 8 | — | 22 | 66 | 4 |
| 2-b2 | | | | | | | |
| Comparative composition | — | — | — | 2 | 28 | 66 | 4 |
| 3-b1 | | | | | | | |
| Comparative composition | — | — | — | 8 | 22 | 66 | 4 |
| 3-b2 | | | | | | | |
| Exemplary composition | 2 | | | | 38 | 56 | 4 |
| 1-c1 | | | | | | | |
| Exemplary composition | 10 | | | | 30 | 56 | 4 |
| 1-c2 | | | | | | | |
| Comparative composition | | 2 | | | 38 | 56 | 4 |
| 1-c1 | | | | | | | |
| Comparative composition | | 10 | | | 30 | 56 | 4 |
| 1-c2 | | | | | | | |
| Comparative composition | | | 2 | | 38 | 56 | 4 |
| 2-c1 | | | | | | | |
| Comparative composition | | | 10 | | 30 | 56 | 4 |
| 2-c2 | | | | | | | |
| Comparative composition | | | | 2 | 38 | 56 | 4 |
| 3-c1 | | | | | | | |
| Comparative composition | | | | 10 | 30 | 56 | 4 |
| 3-c2 | | | | | | | |

### Example 2. Stability Analysis of Water-In-Oil Emulsion Composition

In order to evaluate the stability of the water-in-oil emulsion compositions obtained in Example 1, the following experiments were conducted.

### Test Methods:

### A. High Temperature Treatment

The respective water-in-oil emulsion composition was placed in an oven at 50° C. for 3 months. Afterwards, the water-in-oil emulsion composition was taken out and shaken well, followed by visually observing when the water-in-oil emulsion composition separated into an oil layer and an aqueous layer.

### B. Stability Characteristics at Room Temperature

The respective water-in-oil emulsion composition was shaken well, and was then placed at room temperature for 1 minute, followed by visually observing when the water-in-oil emulsion composition separated into an oil layer and an aqueous layer.

### Results:

As shown in Table 2 below, after the high temperature treatment at 50° C. for 3 months, the exemplary compositions 1-a1, 1-a2, 1-b1, 1-b2, 1-c1, and 1-c2 could be homogenized, and these exemplary compositions separated into oil and aqueous layers after 1 minute. Besides, similar results were observed at room temperature.

In contrast, after the high temperature treatment at 50° C. for 3 months, all the comparative compositions could not be homogenized, and these comparative compositions separated into oil and aqueous layers within 1 minute. Besides, similar results were observed at room temperature.

Summarizing the above test results, it is clear that the water-in-oil emulsion compositions of the present disclosure is effective in exhibiting long-lasting satisfactory stability and heat resistance in the absence of a surfactant and a gelling agent.

TABLE 2

| | | Stable at room |
|---|---|---|
| | Stable at 50° C. | temperature |
| Exemplary composition | + | ∘ |
| 1-a1 | | |
| Exemplary composition | + | ∘ |
| 1-a2 | | |
| Comparative composition | − | x |
| 1-a1 | | |
| Comparative composition | − | x |
| 1-a2 | | |
| Comparative composition | − | x |
| 2-a1 | | |
| Comparative composition | − | x |
| 2-a2 | | |
| Comparative composition | − | x |
| 3-a1 | | |
| Comparative composition | − | x |
| 3-a2 | | |
| Exemplary composition | + | ∘ |
| 1-b1 | | |
| Exemplary composition | + | ∘ |
| 1-b2 | | |
| Comparative composition | − | x |
| 1-b1 | | |
| Comparative composition | − | x |
| 1-b2 | | |
| Comparative composition | − | x |
| 2-b1 | | |
| Comparative composition | − | x |
| 2-b2 | | |
| Comparative composition | − | x |
| 3-b1 | | |
| Comparative composition | − | x |
| 3-b2 | | |
| Exemplary composition | + | ∘ |
| 1-c1 | | |
| Exemplary composition | + | ∘ |
| 1-c2 | | |
| Comparative composition | − | x |
| 1-c1 | | |
| Comparative composition | − | x |
| 1-c2 | | |
| Comparative composition | − | x |
| 2-c1 | | |
| Comparative composition | − | x |
| 2-c2 | | |
| Comparative composition | − | x |
| 3-c1 | | |
| Comparative composition | − | x |
| 3-c2 | | |

“+”: The water-in-oil emulsion composition could be homogenized and the stratification occurred after 1 minute.
“−”: The water-in-oil emulsion composition could not be homogenized and the stratification occurred within 1 minute.
“∘”: The stratification occurred after 1 minute.
“x”: The stratification occurred within 1 minute.

### Example 3. Preparation of Sunscreen Lotion Containing Water-In-Oil Emulsion Composition

A sunscreen lotion containing the water-in-oil emulsion composition of the present disclosure was prepared using the recipe shown in Table 3 and according to the procedures described in Example 1. The thus obtained sunscreen lotion was subjected to stability analysis according to the test methods described in Example 2.

It was found that after the high temperature treatment at 50° C. for 3 months, the sunscreen lotion could be homogenized, and the sunscreen lotion separated into oil and aqueous layers after 1 minute.

Besides, similar results were observed at room temperature. This result indicates that the water-in-oil emulsion composition of the present disclosure can be formulated into a skincare product (e.g. a hand cream, a nutrient cream, or a sunscreen lotion), and such skincare product can exhibit excellent stable characteristics and storability.

TABLE 3

| | Ingredients | Amount (wt %) |
|---|---|---|
| Oil phase | Avena sativa kernel oil | 7 |
| | Octvidodecanol | 25 |
| | Titanium dioxide (and) | 10 |
| | alumina (and) stearic | |
| | acid (Eusolex ® T-S, | |
| | Merck KGaA Inc.) | |
| | Silica (and) CI 77891 | 2 |
| | (and) CI 77491 | |
| | (RonaFlair ® Flawless, | |
| | Merck KGaA Inc.) | |
| Aqueous phase | Water | 50.47 |
| | Pentylene glycol | 3 |
| | Aqua (and) glycerin | 2 |
| | (and) sodium levulinate | |
| | (and) sodium anisate | |
| | (Dermosoft ® 1388 ECO, | |
| | Evonik Dr. Straetmans | |
| | GmbH) | |
| | Citric acid | 0.03 |
| | Sodium chloride | 0.5 |

All patents and literature references cited in the present specification, as well as the references described therein, are hereby incorporated by reference in their entirety. In case of conflict, the present description, including definitions, will prevail.

While the disclosure has been described in connection with what are considered the exemplary embodiments, it is understood that this disclosure is not limited to the disclosed embodiments but is intended to cover various arrangements included within the spirit and scope of the broadest interpretation so as to encompass all such modifications and equivalent arrangements.

What is claimed is:

1. A water-in-oil emulsion composition comprising, based on the total weight of the water-in-oil emulsion composition:
2 wt % to 10 wt % of Avena sativa kernel oil;
15 wt % to 38 wt % of octyldodecanol; and
water;
the water-in-oil emulsion composition being free from a surfactant and a gelling agent.

2. The water-in-oil emulsion composition according to claim 1, wherein the Avena sativa kernel oil contains 0.5 wt % to 2 wt % of a ceramide, based on the total weight of the Avena sativa kernel oil.

3. The water-in-oil emulsion composition according to claim 1, further comprising a preservative.

4. The water-in-oil emulsion composition according to claim 3, wherein the preservative is present in an amount ranging from 2 wt % to 4 wt %, based on the total weight of the water-in-oil emulsion composition.

5. The water-in-oil emulsion composition according to claim 3, wherein the preservative is selected from the group consisting of Lactobacillus/radish root ferment filtrate, levulinic acid, sodium levulinate, sodium citrate, sodium anisate, glycerol, and combinations thereof.
